(19) United States
(12) Patent Application Publication
(10) Pub. No.: US 2002/0054927 A1
(43) Pub. Date: May 9, 2002

(54) Process for the extraction of an active principle from leaves of Olea Europaea to promote the synthesis of stress proteins, obtained active principle

(76) Inventor: Jean-Jacques Paufique, Objat (FR)

Correspondence Address: YOUNG & THOMPSON, 745 SOUTH 23RD STREET 2ND FLOOR, ARLINGTON, VA, 22202

(21) Appl. No.: 09/947,572

(22) Filed: Sep. 7, 2001

(30) Foreign Application Priority Data
Sep. 7, 2000 (FR) 00 11398

(51) Int. Cl. A61K 35/78

(52) U.S. Cl. 424/769; 424/774

(57) ABSTRACT

The object of the invention is a process for the extraction of an active principle for improvement for the production of stress proteins particularly to guard against the effect of ultraviolet radiation, characterized in that it comprises the following steps:

solubilization of powder of dried olive leaves (Olea Europaea) in water for 2 hours at 60° C.,

decantation, and

filtration to separate the active phase, concentration, and

sterilizing filtration on a membrane so as to limit the presence of microorganisms, of total mesophilic flora, of yeast, and of molds.

The invention also covers a cosmetic composition and a process for improving the production of stress proteins.

The present invention relates to a process for the extraction of an active principle from Olea Europaea to promote the synthesis of stress proteins, as well as the obtained active principle and cosmetic compositions including this active principle.

In the field of cosmetics particularly, there is interest in all active principles adapted to give rise to reparative activity of the cells.

More particularly as to the skin, it is known to seek active principles which permit acting on the cells of the skin when the latter are damaged by UV radiation.

The stress proteins known under the name HSP (Heat-Shock Proteins), are proteins naturally present in the cells of living organisms. These proteins are produced in response to stresses caused by heat, certain toxic metabolites, oxidative stress, UV radiation.

These proteins are also called chaperone molecules because they play a protective role for the other proteins about the cell.

If a cell is exposed to stress, the cell responds by increasing its production of chaperone molecules to protect the vital proteins.

Thus one of the first functions attributed to these chaperone molecules is the protection of the cutaneous cells subjected to solar radiation.

The stress proteins thus permit suppressing deleterious radicals and above all permit performing cell functions and restoring the metabolic functions to basic values.

These stress proteins have beneficial effects but their production should be limited in the face of long lasting stress to permit achieving equilibrium in the cell because an excess of such proteins gives rise also to the death of said cell.

In protein families such as have been classified according to their molecular weight and/or their sequences, tests permitting characterization of the active principle have related to stress proteins called HSP70 and HSP32.

The invention will be described hereafter, in connection with an active principle extracted from leaves of Olea Europaea, in connection with the accompanying drawings, which show:

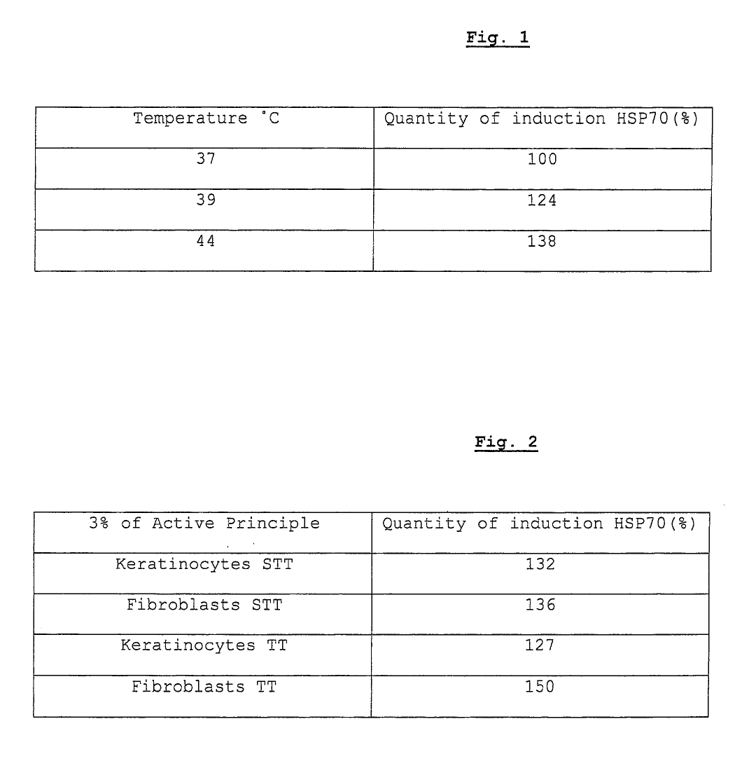

FIG. 1, a recapitulative table of the effects of temperature on the production of stress proteins HSP 70,

FIG. 2, a table of the effects of the active principle according to the present invention on the production of stress proteins HSP 70 in human keratinocyte and fibroblast cultures, with and without thermal treatment,

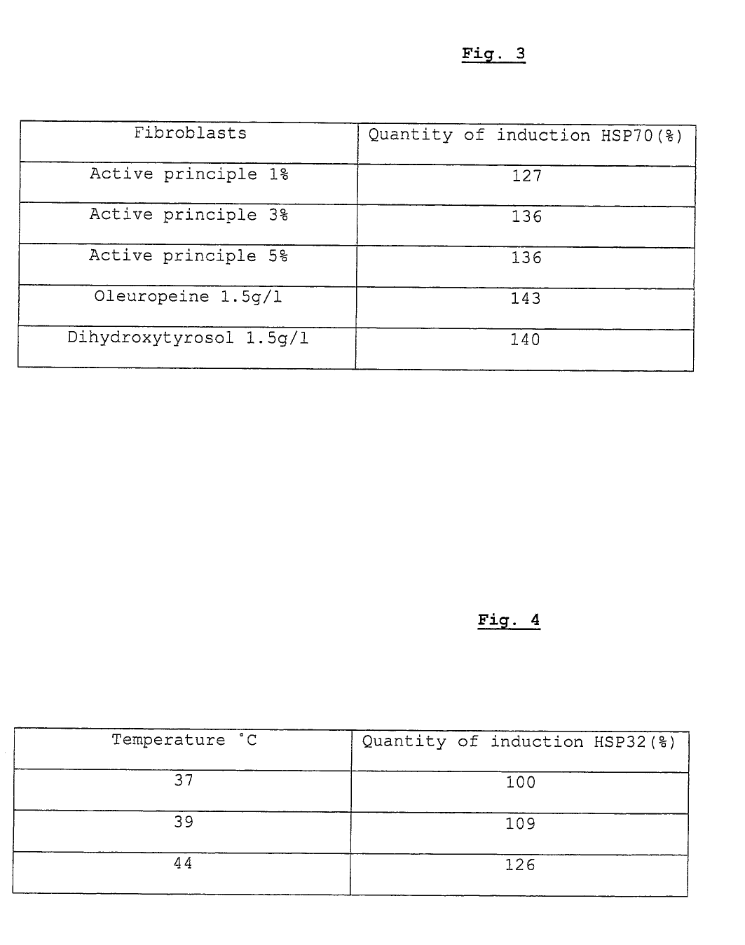

FIG. 3, a table of the effects-dosages of the active principle on the induction of stress proteins HSP 70,

FIG. 4, a recapitulative table of the effects of temperature on the production of stress proteins HSP 32,

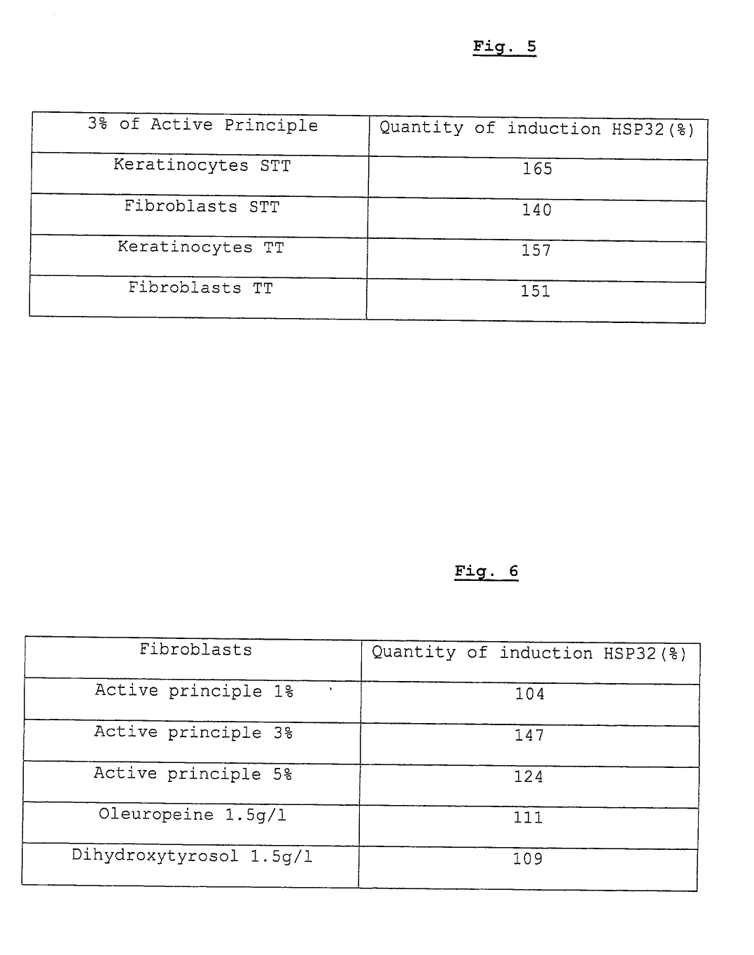

FIG. 5, a table of the effects of the active principle according to the present invention on the production of stress proteins HSP 32 in human keratinocyte and fibroblast cultures, with and without thermal treatment, and

FIG. 6, a table of the effects-dosages of the active principle on the induction of stress proteins HSP 32.

1/Extraction Process.

The active principle according to the present invention is obtained from the leaves of Olea Europaea whose extraction protocol comprises the following series of steps:

solubilization of a powder of dried leaves of Olea Europaea, in water, for 2 hours at 60° C.,

decantation, and

filtration so as to separate the active phase, concentration, and

sterilizing filtration on a membrane so as to limit the presence of microorganisms, of total mesophilic flora, of yeast, and of molds.

2/Characterization of the Active Principle:

The active principle thus obtained is characterized by the following analytical parameters:

Dry Material:

Quantity of dry material: comprised between 10 and 50 g/l, more particularly between 20 and 40 g/l, principally between 25 and 35 g/l. This quantity is obtained by stoving at 105° C. until a constant weight is obtained,

pH:

The value of the pH is comprised between 3.0 and 10.0, more particularly between 4.0 and 7.0, particularly between 4.5 and 5.5, obtained by the potentiometric method at ambient temperature.

Total Polyphenols:

The phenolic compounds form colored complexes in the presence of potassium ferricyanide, whose intensity can be detected at 715 nm. This permits determining the quantity of phenolic compounds over a standard curve. This curve is obtained from a standard range of gallic acid from 40 to 120 mg/l.

The content of phenolic compounds is thus expressed in mg of gallic acid per liter of active material.

The obtained value is comprised between 500 and 5000 mg/l, more particularly between 500 and 3000 mg/l, particularly between 500 and 2000 mg/l.

Total Sugars

The material used for the determination of the total sugar content is the DUBOIS method (DUBOIS, M et al., (1956) Analytical chemistry, 28, No. 3, pp. 350-356).

In the presence of phenol and concentrated sulfuric acid, the reducing sugars give a yellow-orange colored compound and the spectrophotometric reading gives a value which, from the curve DO=f(conc) obtained by standards (comprising ⅓ mannose, ⅓ glucose and ⅓ galactose), permits deducing the value of total sugars for the active principle from leaves of Olea Europaea greater than 2 g/l and more precisely comprised between 2 and 30 g/l, particularly between 6 and 15 g/l.

The bacteriological analysis should show that the total mesophilic flora is less than 100 germs/g and the absence of yeast and molds.

3/Experimentation and determination of the effects on the production of stress proteins

Stress proteins are expressed particularly by the cutaneous cell cultures such as fibroblasts and keratinocytes and the studies which will follow permit determining the effects of the active principle according to the present invention on the synthesis of stress proteins induced by various stresses.

The two families of stress proteins studied are:

HSP 70: this major protein of the family of HSP 70 is the most easily induced by stress, and it is engaged in the processes of thermoresistance and thermoprotection as well as in the mechanisms of protection against induced UVB damage.

The cutaneous quantity of this protein decreases with age at the same time that the capacity to respond to environmental aggressions decreases.

HSP 32: This enzyme is heme-oxygenase-1, which oxidatively cleaves heme which is a pro-oxidant molecule of carbon monoxide and biliverdine. The inductible form of this enzyme is assimilated to a stress protein and the molecular mass by electrophoresis has given its appellation HSP 32.

These two stress proteins play an important role in solar protection and in the repair of damage arising from UV radiation.

HSP 70

1^{st} test with thermal treatment:

At J0, cutaneous cells, fibroblasts and/or keratinocytes are seeded.

At J4, 3% of active principle was added and after an incubation of 30 minutes thermal treatment is carried out for 1 hour at 39° C.

The positive specimen is treated for 1 hour at 44° C.

The total RNA is extracted. The RNA is reverse-transcripted and the complementary DNA is analyzed by using complementary oligonucleotides of a sequence coding the genes and proteins studied.

The RNAm of β-actine is analyzed for each experimental condition.

By image analysis, using for example the system sold under the name BIO-PROFIL, the band intensities are determined.

2^{nd} test without thermal treatment:

At J0, cutaneous cells, fibroblasts and/or keratinocytes are seeded.

At J3, 3% of active principle is added and left to incubate for 24 hours at 37° C.

At J4, at the end of this incubation, the cells are recovered.

The total RNA is extracted. The RNA is reverse-transcripted and the complementary DNA is analyzed by using complementary oligonucleotides of a sequence coding the genes and proteins used. The RNAm of p-actine is analyzed for each experimental condition.

Similarly to the above, by image analysis, the intensities of the bands are determined.

The percentages are expressed in the ratio of intensity, in which:

$$RE = \frac{RNAm band intensity ( test protein )}{RNAm band intensity ( β \text{-} actine )}$$

$$RT = \frac{RNAm band intensity ( standard protein )}{RNAm band intensity ( β \text{-} actine )}$$

$$\% RNAm \text{/} standard = \frac{RE}{RT} \times 100$$

The results of the thermal treatments of the standard are indicated in the table in FIG. 1, taking as 100% the quantity of RNAm of HSP 70 after incubation at 37° C. of a standard specimen

It is to be noted that it is necessary to reach 44° C. to obtain significant intracellular production of HSP 70 of 138%.

In the table of FIG. 2, there can be compared the results obtained with the test specimen in the case of a Thermal Treatment TT and Without Thermal Treatment STT and with 3% of active principle.

It will be noted that the results obtained even without thermal treatment are significant, because they are substantially identical to those obtained from the standard having been subjected to thermal treatment at 44° C.

It has also been sought to show the effect-dosage of the synthesis of HPS 70 on fibroblasts as well as the recapitulation of the table of FIG. 3. To complete the analysis, there have been carried out tests with Oleuropeine at 1.5 g/l and the basic derivative of oleuropeine, Dihydroxytyrosol, also at 1.5 g/l.

It is seen that Oleuropeine and its basic derivative are very active, which explains the action of the complete active principle. The action of the active principle is optimal from 3%.

HSP 32

The same operative protocol is used but this time the quantity of RNAm of HSP 32 is measured.

The results are shown in FIGS. 4, 5 and 6.

In the case of HSP 32, it is seen that the quantity of induction is improved by thermal treatment (126% for a thermal treatment at 44° C.).

The quantity of induction is on the other hand greatly increased in the presence of the active principle because it reaches 165% in the case of keratinocytes without thermal treatment.

Similarly, the effect-dosage shows that 3% permits achieving a maximum induction, which shows the limited quantity necessary for the effects of the active principle to be optimal.

The invention also covers a cosmetic composition which includes the active principle according to the present invention and which permits increasing the production of stress proteins and guarding against the effects of ultraviolet radiation. This composition comprises at least the active principle of the present invention, or a portion of the active principle concentrated or dried, associated in any suitable galenic form.

The invention also covers the process for production of stress proteins, which consists in disposing preventatively on the skin this cosmetic composition in an amount of at least 0.1% of active principle, preferably 1 to 5%.

1. Process for the extraction of an active principle for improvement of the production of stress proteins particularly to guard against the effect of ultraviolet radiation, characterized in that it comprises the following steps:
solubilization of powders of dried leaves of Olea Europaea, in water for two hours at 60° C.,
decantation, and
filtration so as to separate the active phase, concentration, and
sterilizing filtration on a membrane so as to limit the presence of microorganisms, of total mesophilic flora, of yeast, and of molds.

2. Active principle obtained by the process of claim 1, characterized in that it comprises:
a quantity of dried material: comprised between 10 and 50 g/l more particularly between 20 and 40 g/l principally between 25 and 35 g/l.
pH comprised between 3.0 and 10.0 more particularly between 4.0 and 7.0, particularly between 4.5 and 5.5.
total polyphenols: quantity comprised between 500 and 5000 mg/l, more particularly between 500 and 3000 mg/l, particularly between 500 and 2000 mg/l, expressed as mg of gallic acid.
total sugars: quantity greater than 2 g/l and more precisely comprised between 2 and 30 g/l, particularly between 6 and 15 g/l.

3. Cosmetic composition to increase the production of stress proteins and to guard against the effects of ultraviolet radiation, characterized in that it comprises at least in part the active principle according to claim 2, concentrated or dried, associated in any appropriate galenic form.

4. Process to increase the production of stress proteins characterized in that there is preventatively disposed on the skin a cosmetic composition according to claim 3 in an amount of at least 0.1% of active principle.

5. Process to increase the production of stress proteins according to claim 4, characterized in that between 1 and 5% of active principle is applied.
